(19) United States
(12) Patent Application Publication
(10) Pub. No.: US 2020/0289727 A1
(43) Pub. Date: Sep. 17, 2020

(54) WOUND DRESSING WITH NEGATIVE PRESSURE RETAINING VALVE

(71) Applicant: KCI Licensing, Inc., San Antonio, TX (US)

(72) Inventor: Christopher B. LOCKE, Bournemouth (GB)

(73) Assignee: KCI Licensing, Inc., San Antonio, TX (US)

(21) Appl. No.: 16/769,323

(22) PCT Filed: Dec. 4, 2018
(86) PCT No.: PCT/US2018/063873

§ 371 Date: Jun. 3, 2020

Related U.S. Application Data
(60) Provisional application No. 62/595,289, filed on Dec. 6, 2017.

(51) Int. Cl. A61M 1/00; A61M 39/24; A61F 13/02

(52) U.S. Cl. CPC A61M 1/0088; A61M 1/0052; A61M 1/0086; A61M 2205/7536; A61F 13/0216; A61M 2205/071; A61M 2205/3341; A61M 39/24

(57) ABSTRACT

A wound dressing includes a cover layer, a tube, and a one-way valve. The cover layer is sealable to skin surrounding a wound site and includes a port extending through the cover layer. The tube includes a first end coupled to the cover layer via the port and a second end including an in-line connector configured to releasably attach the tube to a negative pressure source and detach the tube from the negative pressure source. The one-way valve is located along the tube between the first end and the second end and configured to allow fluid flow through the tube in a first direction from the first end to the second end and prevent fluid flow through the tube in a second direction from the second end to the first end.

### CROSS-REFERENCE TO RELATED APPLICATIONS

This application claims the benefit of priority to U.S. Provisional Application No. 62/595,289, filed on Dec. 6, 2017, which is incorporated herein by reference in its entirety.

### BACKGROUND

The present disclosure relates generally to a wound dressing and more particularly to a wound dressing for use as part of a negative pressure wound therapy system.

Negative pressure wound therapy (NPWT) is a type of wound therapy that involves applying a negative pressure to a wound site to promote wound healing. Some wound treatment systems apply negative pressure to a wound site using a pneumatic pump to generate the negative pressure and flow required. However, continuous regulated negative pressure typically requires the pump to remain tethered to the wound site. It would be desirable to provide a wound therapy system and/or a wound dressing that permits additional functionality compared with conventional NPWT systems.

### SUMMARY

One implementation of the present disclosure is a wound dressing including a cover layer, a tube, and a one-way valve. The cover layer is sealable to skin surrounding a wound site and includes a port extending through the cover layer. The tube includes a first end coupled to the cover layer via the port and a second end including an in-line connector configured to releasably attach the tube to a negative pressure source and detach the tube from the negative pressure source. The one-way valve is located along the tube between the first end and the second end and configured to allow fluid flow through the tube in a first direction from the first end to the second end and prevent fluid flow through the tube in a second direction from the second end to the first end.

In some embodiments, the one-way valve is located within the in-line connector at the second end of the tube. In some embodiments, the one-way valve is configured to maintain negative pressure within the tube when the tube is detached from the negative pressure source.

In some embodiments, the wound dressing includes a filter configured to prevent liquid within the tube from reaching the one-way valve. In some embodiments, the filter is located within the in-line connector at the second end of the tube.

In some embodiments, the wound dressing includes a hydrophobic filter configured to retain liquid within the wound dressing. In some embodiments, the fluid flow through the tube is airflow.

In some embodiments, the wound dressing includes a pressure indicator configured to measure a pressure within the tube or at the wound site. In some embodiments, the pressure indicator is a mechanical pressure indicator.

In some embodiments, the negative pressure source includes at least one of a motorized pump or a manually-operable pump.

In some embodiments, an internal volume of the tube between the one way valve and the cover layer defines a negative pressure reservoir fluidly connected with the wound site and configured to stabilize changes in pressure at the wound site. In some embodiments, the negative pressure reservoir within the tube has a volume of at least 5,000 mm³. In some embodiments, the negative pressure reservoir within the tube has a volume of at least 10,000 mm³.

In some embodiments, the negative pressure reservoir is configured to maintain pressure at the wound site at least 50 mmHg below atmospheric pressure when the tube is detached from the negative pressure source. In some embodiments, the negative pressure reservoir is configured to maintain pressure at the wound site at least 50 mmHg below atmospheric pressure for at least 24 hours when the tube is detached from the negative pressure source. In some embodiments, the negative pressure reservoir is configured to prevent pressure at the wound site from changing by more than 75 mmHg within a 12 hour period when the tube is detached from the negative pressure source.

In some embodiments, the tube has a length of at least 1 m. In some embodiments, the tube has an inner diameter between 2 mm and 3 mm. In some embodiments, the tube has an inner diameter between 3 mm and 4 mm. In some embodiments, the tube has an outer diameter of at least 4 mm and a wall thickness of at least 0.75 mm.

In some embodiments, the tube is configured to configured to retract to form a substantially planar coil when not connected to the negative pressure source. In some embodiments, the tube is pre-coiled such that the tube forms a substantially planar coil in the absence of external force applied to the tube.

In some embodiments, a cross-section of the tube is substantially rectangular. In some embodiments, the tube has a wall thickness that provides sufficient rigidity to prevent collapse of the tube when a pressure within the tube is at least 100 mmHg below atmospheric pressure.

Another implementation of the present disclosure is a negative pressure wound therapy (NPWT) system including a negative pressure source, a wound dressing, a tube, and a one-way valve. The wound dressing is sealable to skin surrounding a wound site. The tube includes a first end coupled to the wound dressing and a second end including an in-line connector configured to releasably attach the tube to the negative pressure source and detach the tube from the negative pressure source. The one-way valve is located along the tube and configured to prevent fluid flow through the tube from the second end to the first end.

In some embodiments, the one-way valve is located within the in-line connector at the second end of the tube. In some embodiments, the one-way valve is configured to maintain negative pressure within the tube when the tube is detached from the negative pressure source.

In some embodiments, the NPWT system includes a filter configured to prevent liquid within the tube from reaching the one-way valve. In some embodiments, the filter is located within the in-line connector at the second end of the tube.

In some embodiments, the wound dressing includes a hydrophobic filter configured to retain liquid within the wound dressing. In some embodiments, the fluid flow through the tube is airflow.

In some embodiments, the NPWT system includes a pressure indicator configured to measure a pressure within the tube or at the wound site. In some embodiments, the pressure indicator is a mechanical pressure indicator.

In some embodiments, the negative pressure source includes at least one of a motorized pump or a manually-operable pump.

In some embodiments, an internal volume of the tube between the one way valve and the wound dressing defines a negative pressure reservoir fluidly connected with the wound site and configured to stabilize changes in pressure at the wound site.

In some embodiments, the negative pressure reservoir within the tube has a volume of at least 5,000 mm³. In some embodiments, the negative pressure reservoir within the tube has a volume of at least 10,000 mm³.

In some embodiments, the negative pressure reservoir is configured to maintain pressure at the wound site at least 50 mmHg below atmospheric pressure when the tube is detached from the negative pressure source. In some embodiments, the negative pressure reservoir is configured to maintain pressure at the wound site at least 50 mmHg below atmospheric pressure for at least 24 hours when the tube is detached from the negative pressure source. In some embodiments, the negative pressure reservoir is configured to prevent pressure at the wound site from changing by more than 75 mmHg within a 12 hour period when the tube is detached from the negative pressure source.

In some embodiments, the tube has a length of at least 1 m. In some embodiments, the tube has an inner diameter between 2 mm and 3 mm. In some embodiments, the tube has an inner diameter between 3 mm and 4 mm. In some embodiments, the tube has an outer diameter of at least 4 mm and a wall thickness of at least 0.75 mm.

In some embodiments, the tube is configured to configured to retract to form a substantially planar coil when not connected to the negative pressure source. In some embodiments, the tube is pre-coiled such that the tube forms a substantially planar coil in the absence of external force applied to the tube.

In some embodiments, a cross-section of the tube is substantially rectangular. In some embodiments, the tube has a wall thickness that provides sufficient rigidity to prevent collapse of the tube when a pressure within the tube is at least 100 mmHg below atmospheric pressure.

Another implementation of the present disclosure is a wound dressing including a cover layer, a tube, a one-way valve, a filter, and a pressure indicator. The cover layer is sealable to skin surrounding a wound site and includes a port extending through the cover layer. The tube includes a first end coupled to the cover layer via the port and a second end including an in-line connector configured to releasably attach the tube to a negative pressure source and detach the tube from the negative pressure source. The one-way valve is located within the in-line connector and configured to prevent fluid flow into the tube via the second end. The filter is located within the in-line connector and configured to prevent liquid within the tube from reaching the one-way valve. The pressure indicator is located within the in-line connector and configured to measure a pressure at the second end of the tube.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is an exploded view of a negative pressure wound therapy (NPWT) system including a wound dressing, a tube, and a pump unit, according to an exemplary embodiment.

FIG. 2 is a perspective view of the pump unit of FIG. 1, according to an exemplary embodiment.

FIG. 3 is a top perspective view of the wound dressing of FIG. 1, according to an exemplary embodiment.

FIG. 4 is another top view of the wound dressing of FIG. 1 illustrating the tube retracted into a coil, according to an exemplary embodiment.

FIG. 5 is a cross-sectional view of the tube of FIG. 1 illustrating an embodiment in which the tube has a circular cross-section, according to an exemplary embodiment.

FIG. 6 is a cross-sectional view of the tube of FIG. 1 illustrating an embodiment in which the tube has a rectangular cross-section, according to an exemplary embodiment.

FIG. 7 is a block diagram of the NPWT system of FIG. 1, according to an exemplary embodiment.

FIG. 8 is a graph illustrating results of a dry test experiment performed using the wound dressing of FIG. 1, according to an exemplary embodiment.

FIG. 9 is a graph illustrating results of a wet test experiment performed using the wound dressing of FIG. 1, according to an exemplary embodiment.

### DETAILED DESCRIPTION

### Overview

Referring generally to the FIGURES, a wound dressing and negative pressure wound therapy system are shown, according to various exemplary embodiments. Negative pressure wound therapy (NPWT) is a type of wound therapy that involves applying a negative pressure to a wound site (relative to atmospheric pressure) to promote wound healing. The NPWT system includes a negative pressure source (e.g., a manually-operable or motorized pump), a wound dressing, and a tube connecting the negative pressure source to the wound dressing. The wound dressing is sealable to a patient's skin surrounding a wound site. The pump operates to create negative pressure at the wound site by removing air from the wound site via the tube.

The tube includes a first end coupled to the wound dressing and a second end opposite the first end. The second end of the tube includes an in-line connector configured to releasably attach the tube to the negative pressure source and detach the tube from the negative pressure source. The in-line connector includes a one-way valve configured to prevent fluid flow through the tube from the second end to the first end. Accordingly, the one-way valve allows the tube to be disconnected from the negative pressure source without losing the negative pressure within the tube and/or at the wound site. The internal volume of the tube also acts as a negative pressure reservoir to stabilize changes in pressure at the wound site. These and other features of the wound dressing and NPWT system are described in greater detail below.

### Negative Pressure Wound Therapy (NPWT) System & Wound Dressing

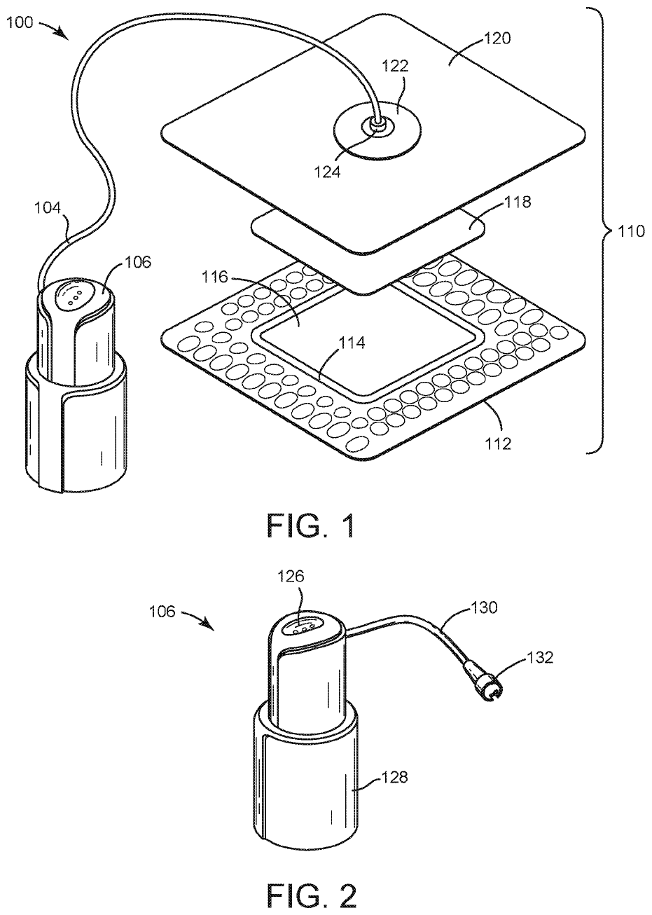

Referring now to FIG. 1, a negative pressure wound therapy system 100 is shown, according to an exemplary embodiment. System 100 is shown to include a wound dressing 110, pump unit 106, and a tube 104 connecting wound dressing 110 with pump unit 106. Wound dressing 110 can be sealed to a patient's skin surrounding a wound site and may provide an airtight seal over the wound site. Pump unit 106 can be configured to draw air or other fluids from the wound site via tube 104 such that the wound site is maintained at negative pressure relative to atmospheric pressure. In various embodiments, pump unit 106 can be a manually-operable pump, a motorized pump, or any other device that functions as a negative pressure source.

Wound dressing 110 can be formed as a substantially flat sheet for topical application to wounds or contoured for application to body surfaces having high curvature. The size of wound dressing 110 can vary depending on the size of the wound to be dressed. For example, it is contemplated that the size of wound dressing 110 can range from 1 cm² to 200 cm², and more preferably from 4 cm² to 100 cm². However, other shapes and sizes of wound dressing 110 are also possible depending on the intended use. Wound dressing 110 is shown to include a cover layer 120, an upper pressure distribution layer 118, an absorbent layer 116, a lower pressure distribution layer 114, and a wound interface layer 112.

Cover layer 120 can be configured to seal to skin surrounding a wound site. In some embodiments, cover layer 120 is made of a material that prevents or greatly reduces the permeation of air or other fluids through cover layer 120. For example, cover layer 120 may be made of polyurethane or other suitable polymeric materials and may include an elastomeric film or membrane that can provide a seal around the wound site. In some embodiments, cover layer 120 provides a barrier to microbes, a barrier to external contamination, and protection from physical trauma. For example, cover layer 120 may be constructed from a material that can reduce pressure losses and provide a fluid seal between two components or two environments, such as between a therapeutic environment between cover layer 120 and the wound and a local external environment.

In some embodiments, cover layer 120 is coated with an acrylic or other adhesive. The adhesive applied to cover layer 120 ensures that wound dressing 110 adheres to the patient's skin and that wound dressing 110 remains in place throughout the wear time. In some embodiments, the perimeter of cover layer 120 extends beyond (e.g., circumscribes) the perimeters of upper pressure distribution layer 118, absorbent layer 116, lower pressure distribution layer 114, and wound interface layer 112 to provide an adhesive-coated margin for adhering wound dressing 110 to the skin of a patient adjacent to the wound being treated. The adhesive-coated margin may extend around all sides of layers 112-118 such that wound dressing 110 is a so-called island dressing. In other embodiments, the adhesive-coated margin can be eliminated and wound dressing 110 can be adhered to the patient's skin using other techniques.

In some embodiments, cover layer 120 includes a connector pad 122 and a port 124 extending through cover layer 120. Connector pad 122 can be coupled to a first end of tube 104 and may secure the first end of tube 104 to cover layer 120. Tube 104 may extend through cover layer 120 and connector pad 122 via port 124 such that tube 104 is fluidly connected to a space between cover layer 120 and the wound. This allows pump unit 106 to remove air or other fluids from the wound site via port 124 and tube 104 to maintain the space within wound dressing 110 at negative pressure. Upper pressure distribution layer 118 may be located adjacent to cover layer 120 (opposite connector pad 122) and can be configured to distribute the negative pressure across absorbent layer 116. Similarly, lower pressure distribution layer 114 can be configured to distribute the negative pressure across wound interface layer 112 and ultimately the surface of the wound.

Absorbent layer 116 may be located between pressure distribution layers 114 and 118 and can be configure to absorb wound exudate or other fluids at the wound site. In some embodiments, absorbent layer 116 includes a superabsorbent material such as a hydrogel or hydrogel composition. Several examples of hydrogels and hydrogel compositions which can be used to form absorbent layer 116 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

The expressions “hydrogel” and “hydrogel compositions” used herein are not to be considered as limited to gels which contain water, but extend generally to all hydrophilic gels and gel compositions, including those containing organic non-polymeric components in the absence of water. For example, absorbent layer 116 may be formed from a polyurethane that entraps water to form a gel. In some embodiments, absorbent layer 116 is substantially continuous and/or substantially non-porous or non-foamed. Absorbent layer 116 may include a flexible plasticized hydrophilic polymer matrix having a substantially continuous internal structure. The density of absorbent layer 116 may be greater than 0.5 g/cm³, more preferably greater than 0.8 g/cm³, and most preferably from 0.9 to 1.1 g/cm³. In some embodiments, the thickness of absorbent layer 116 is from 1 mm to 10 mm, more preferably from 2 mm to 5 mm.

In some embodiments, absorbent layer 116 is cross-linked and preferably it is substantially insoluble in water at ambient temperature. However, the structure of absorbent layer 116 absorbs and entraps liquid to provide a highly hydrated gel structure in contrast to the porous foam structure absorbent layer 116. Preferably, the gel can absorb 1 to 10 g/g of physiological saline at 20°, more preferably 2 to 5 g/g.

In some embodiments, the dry weight of absorbent layer 116 is from 1000 to 5000 g/m², more preferably from 2000 to 4000 g/m². In some embodiments, absorbent layer 116 includes from 1% to 30% of water, more preferably from 10% to 20% by weight of water before use. In some embodiments, absorbent layer 116 contains from 1% to 40%, more preferably from 5 to 15%, by weight of one or more humectants, preferably selected from the group consisting of glycerol, propylene glycol, sorbitol, mannitol, polydextrose, sodium pyrrolidine carboxylic acid (NaPCA), hyaluronic acid, aloe, jojoba, lactic acid, urea, gelatin, lecithin and mixtures thereof. The entrapped water and optional humectants give the hydrogel a soft, moist wound-friendly surface for contacting the wound.

In some embodiments, absorbent layer 116 includes a hydrophilic foam. The hydrophilic foam can be laminated or otherwise coupled to the superabsorbent material via a fusible fiber. The hydrophilic foam may include a polyurethane foam and/or a flexible plasticized hydrophilic polymer matrix having an internal cellular structure. Several examples of hydrophilic foams which can be used to in absorbent layer 116 are described in detail in U.S. Pat. No. 8,097,272 issued Jan. 17, 2012, U.S. Pat. No. 8,664,464 issued Mar. 4, 2014, and U.S. Pat. No. 8,058,499 issued Nov. 15, 2011. The entire disclosure of each of these patents is incorporated by reference herein.

Advantageously, the hydrophilic foam provides enhanced absorbency for liquid exudate. This is because the initial substantially anhydrous condition and porous structure of the hydrophilic foam enables it to absorb a larger amount of water by both chemical and physical absorption that is the case for the corresponding hydrogel material. Furthermore, the porous structure of the foam provides for rapid uptake of liquid exudate, in contrast to pure hydrogel dressings.

In some embodiments, the hydrophilic foam layer has a thickness of from 1 to 20 mm, more preferably from 1.5 to 5 mm. In some embodiments, the hydrophilic foam has a density of from 0.28 g/cm³ to 0.5 g/cm³, and more preferably from 0.32 g/cm³ to 0.48 g/cm³. Preferably, the hydrophilic foam has an elongation to break of at least 150%, more preferably from 500% to 1000%. The hydrophilic foam can absorb aqueous fluids such as wound exudate with swelling. The hydrophilic foam may be highly cross-linked and substantially insoluble in water.

In some embodiments, the hydrophilic foam has an absorbency of at least 3 grams of saline per gram of foam, and preferably a swellability in water of at least 200%. In some embodiments, the hydrophilic foam is constructed using the foam as described in European Patent No. 0541391 issued Jun. 10, 1998, the entire disclosure of which is incorporated by reference herein. In some embodiments, the hydrophilic foam includes less than 10% water prior to use as an absorbent, more preferably less than 5% water, and even more preferably it contains less than 2% of water before use.

Wound interface layer 112 may form a wound-contacting surface of wound dressing 110. In some embodiments, wound interface layer 112 is made of silicone or other non-adherent materials that provides an effective seal for negative pressure, yet enable easy repositioning or removal, minimising trauma to periwound skin. Wound interface layer 112 can be configured to reduce potential adherence of absorbent layer 116 or lower pressure distribution layer 114 to the wound or tissue site, to enable fluid to be effectively drawn away from the wound via wound interface layer 112, absorbent layer 116, or both. In some embodiments, wound interface layer 112 is made of a hydrophobic material such as polyethylene (PE) or other hydrophobic polymers. The use of a hydrophobic material for wound interface layer 112 may be particularly advantageous to prevent the attachment of bacteria to the wound or tissue site. In some embodiments, wound interface layer 112 is perforated for increased fluid flow.

In various embodiments, wound interface layer 112 may include at least one of an alkyl acrylate polymer (e.g., a methyl acrylate polymer, an ethyl acrylate polymer, or the like) an alkacrylate polymer (e.g., a methacrylate polymer, an ethacrylate polymer, or the like) and/or an alkyl alkacrylate polymer (e.g., a methyl methacrylate polymer, an ethyl methacrylate polymer, a methyl ethacrylate polymer, an ethyl ethacrylate polymer, or the like). Such (alk)acrylate polymers may be homopolymers but are more often copolymers, for example, with olefin comonomers. In some embodiments, wound interface layer 112 includes anethylene-methyl acrylate copolymer, such as used in TIELLE dressings and SILVERCEL non-adherent dressings available from Systagenix Wound Management, Limited. In various embodiments, wound interface layer 112 may include a silicone or polysiloxane polymer or copolymer.

In some embodiments, wound dressing 110 includes a hydrophobic filter configured to retain liquids within wound dressing 110. Such liquids may include, for example, wound exudate, water, condensed fluid, and/or other liquids. The hydrophobic filter may prevent any liquids within wound dressing 110 from leaving wound dressing 110 and entering tube 104. Accordingly, any fluid flow through tube 104 may be limited to airflow (or other gasses) in some embodiments.

### Pump Unit

Referring now to FIG. 2, pump unit 106 is shown in greater detail, according to an exemplary embodiment. Pump unit 106 is shown as a manually-operable pump having a plunger 126 and a shell 128. Plunger 126 can be aligned with a central axis of shell 128 and configured to move axially relative to shell 128. A user can press the top surface of plunger 126 toward shell 128 to cause plunger 126 to retract into shell 128. In some embodiments, plunger 126 is coupled to a spring within shell 128 that causes plunger 126 to return to an extended position (as shown in FIG. 2) in the absence of external force.

Plunger 126 and shell 128 may house an internal pneumatic chamber that decreases in volume when plunger 126 is depressed and increases in volume when plunger 126 is extended. The pneumatic chamber may be pneumatically connected to the atmosphere outside of pump unit 106 via a one-way valve that allows air to exit the pneumatic chamber but prevents air from entering the pneumatic chamber via the one-way valve. Accordingly, when plunger 126 is depressed, the air within the pneumatic chamber may be forced through the one-way valve and discharged to the atmosphere outside pump unit 106. When plunger 126 is released, air from tube 130 may be drawn into pump unit 106. A first end of tube 130 may connect to plunger 126, whereas a second end of tube 130 may include a connector 132. Connector 132 can be configured to attach to an in-line connector of tube 104 (described in greater detail below) to couple pump unit 106 to wound dressing 110. Accordingly, pump unit 106 can be operated to draw air from wound dressing 110 to provide negative pressure within wound dressing 110.

Although pump unit 106 is shown as a manually-operable pump, it should be understood that any other types of pump can be used to provide a similar effect. For example, pump unit 106 may be a motorized pump or any other type of device that functions as a negative pressure source. A “negative pressure source” is any type of pump or other device that operates to create a negative pressure zone or space relative to the pressure of the local environment (e.g., atmospheric pressure) around surrounding wound dressing 110. The negative pressure source can be coupled to wound dressing 110 via tube 104 to maintain the wound site negative pressure, thereby providing negative pressure wound therapy at the wound site.

Tube with in-Line Connector

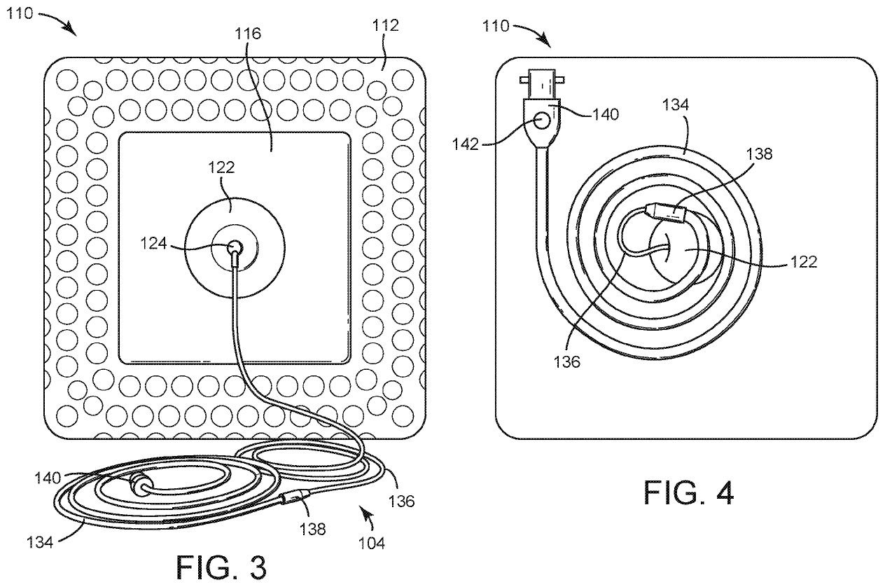

Referring now to FIGS. 3-4, tube 104 is shown in greater detail, according to an exemplary embodiment. A first end of tube 104 can be coupled to wound dressing 110 via connector pad 122 and/or cover layer 120. A second end of tube 104 includes an in-line connector 140. In-line connector 140 is configured to releasably attach tube 104 to pump unit 106 (or another negative pressure source) and detach tube 104 from pump unit 106 (or another negative pressure source). Advantageously, in-line connector 140 may include an internal one-way valve 146 (shown in FIG. 7) configured to allow air to exit tube 104 but prevent air from entering tube 104. Accordingly, the negative pressure within tube 104 may be maintained when tube 104 is detached from the negative pressure source.

In some embodiments, tube 104 includes a wide portion 134 and a narrow portion 136 linked by a reduction component 138. Wide portion 134 may have a relatively larger diameter and/or cross-sectional area than narrow portion 136. In some embodiments, in-line connector 140 is located at a free end of wide portion 134, whereas narrow portion 136 is coupled to wound dressing 110. In some embodiments, tube 104 has a length of at least one meter. However, it is contemplated that tube 104 can have any other length in various other embodiments.

Referring particularly to FIG. 4, in-line connector 140 is shown to include a pressure indicator 142. Pressure indicator 142 can be configured to measure air pressure within tube 104 at the location of in-line connector 140. The pressure measured by pressure indicator 142 may also be the pressure at the wound site due to the pneumatic coupling provided by tube 104. In some embodiments, pressure indicator 142 is a mechanical pressure indicator. For example, pressure indicator 142 may include a sealed chamber configured to expand and contract responsive to the pressure within tube 104. The sealed chamber can be coupled to a colored slider or other visual indicator that moves over a window when the sealed chamber expands and contracts. Accordingly, the portion of the colored slider or other indicator visible through the window may indicate the pressure within tube 104. In other embodiments, pressure indicator 142 may be an electronic pressure sensor or any other type of pressure indicator.

Tube 104 can be configured to configured to retract to form a substantially planar coil when not connected to the negative pressure source. This allows tube 104 to retract into a compact arrangement when disconnected from the negative pressure source. Applying an external force to tube 104 may cause tube 104 to extend from the compact arrangement shown in FIG. 4 to allow in-line connector 140 to reach a negative pressure source. In some embodiments, tube 104 is pre-coiled (e.g., via a heat treatment) such that tube 104 forms a substantially planar coil in the absence of the external force.

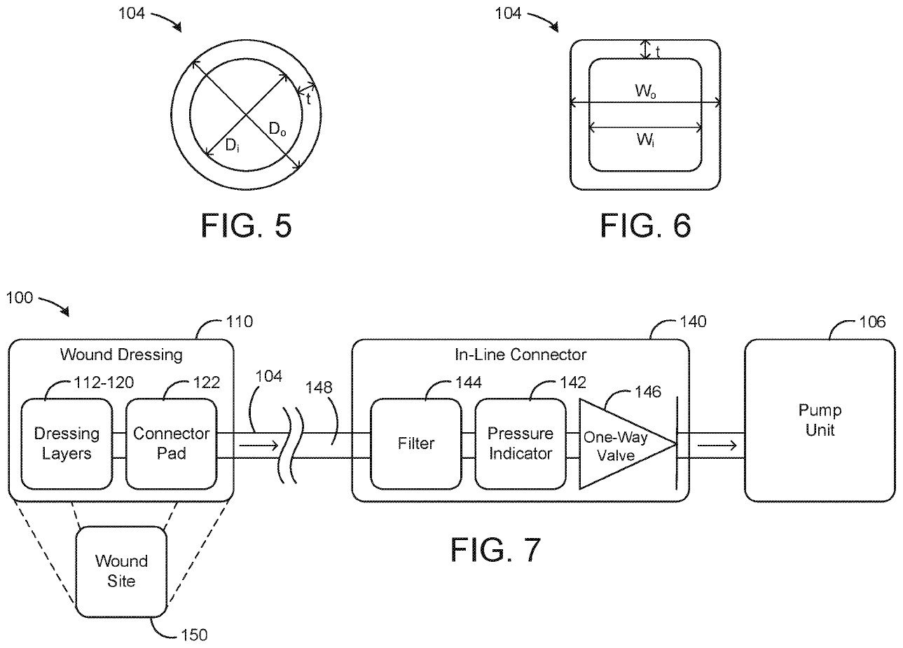

Referring now to FIGS. 5-6, tube 104 can have a variety of different cross-sectional shapes and sizes. FIG. 5 illustrates an embodiment in which tube 104 has a substantially circular cross-section. In some embodiments, tube 104 has an inner diameter (Dᵢ) between 2 mm and 3 mm. In other embodiments, tube 104 has an inner diameter (Dᵢ) between 3 mm and 4 mm. In some embodiments, tube 104 has an outer diameter (D₀) of at least 4 mm. Tube 104 may have a wall thickness (t) that provides sufficient rigidity to prevent the collapse of tube 104 when the pressure within tube 104 is at least 100 mmHg below atmospheric pressure. For example, tube 104 may have a wall thickness (t) of at least 0.75 mm. However, the wall thickness of tube 104 may vary depending on the material used to form tube 104.

FIG. 6 illustrates an embodiment in which tube 104 has a substantially rectangular cross-section. In some embodiments, tube 104 has an inner width (Wᵢ) between 2 mm and 3 mm. In other embodiments, tube 104 has an inner width (Wᵢ) between 3 mm and 4 mm. In some embodiments, tube 104 has an outer width (Wₒ) of at least 4 mm. When tube 104 has a rectangular cross-section, tube 104 may have a wall thickness (t) that provides sufficient rigidity to prevent the collapse of tube 104 when the pressure within tube 104 is at least 100 mmHg below atmospheric pressure. For example, tube 104 may have a wall thickness (t) of at least 0.75 mm. However, the wall thickness of tube 104 may vary depending on the material used to form tube 104.

Referring now to FIG. 7, a block diagram illustrating several components of NPWT system 100 in greater detail is shown, according to an exemplary embodiment. As discussed above, wound dressing 110 may include several dressing layers 112-120 and a connector pad 122. Wound dressing 110 can be configured to seal to a patient's skin surrounding wound site 150. A first end of tube 104 may be coupled to wound dressing 110 via connector pad 122. In-line connector 140 may be located at a second end of tube 104 opposite the first end.

In-line connector 140 is shown to include a pressure indicator 142, a filter 144, and a one-way valve 146. In some embodiments, pressure indicator 142, filter 144, and one-way valve 146 are located within in-line connector 140. Pressure indicator 142 may be a mechanical pressure indicator, an electronic pressure sensor, or any other type of pressure sensing device, as previously described. Filter 144 can be configured to prevent any liquid within tube 104 from reaching one way valve 146. For example, wound exudate from wound site 150 may be drawn into tube 104 when pump unit 106 operates to draw a negative pressure within tube 104. Filter 144 may be positioned between one-way valve 146 and wound dressing 110 such that any liquid within tube 104 does not reach one-way valve 146 and/or pump unit 106.

Advantageously, one-way valve 146 may permit airflow through one-way valve 146 in the direction of the arrows in FIG. 7 (i.e., from tube 104 to pump unit 106) but may prevent airflow in the opposite direction (i.e., airflow into tube 104 via in-line connector 140). This allows pump unit 106 to remove air from tube 104 when in-line connector 140 is connected to pump unit 106, thereby creating negative pressure within tube 104. However, one-way valve 146 prevents airflow into tube 104, thereby maintaining the negative pressure within tube 104, even when in-line connector 140 disconnected from pump unit 106.

In some embodiments, an internal volume of tube 104 acts as a negative pressure reservoir 148 for wound dressing 110. Negative pressure reservoir 148 may be defined as the volume within tube 104 that can be occupied by air or other fluids and may extend between wound dressing 110 and pump unit 106. Negative pressure reservoir 148 may be maintained at a negative pressure relative to the pressure of the local environment (e.g., atmospheric pressure) outside wound dressing 110. Negative pressure reservoir 148 may be fluidly connected with wound site 150 and may be maintained at the same pressure as wound site 150.

Advantageously, negative pressure reservoir 148 may act to stabilize changes in pressure at wound site 150. For example, wound site 150 may exude liquid over time that occupies some of the open volume within wound dressing 110. If wound dressing 110 were not connected to negative pressure reservoir 148, the decrease in open volume would significantly increase the pressure at wound site 150 and would lessen the therapeutic effects of negative pressure wound therapy. However, because wound dressing 110 is fluidly connected to negative pressure reservoir 148, any loss in open volume within wound dressing 110 may be insignificant relative to the volume of negative pressure reservoir 148. Accordingly, the decrease in open volume within wound dressing 110 may not significantly increase the pressure at wound site 150. Negative pressure reservoir 148 may also stabilize changes in pressure caused by air leakage into wound dressing 110 and/or tube 104 in a similar manner.

The degree of pressure stabilization provided by negative pressure reservoir 148 may depend on the volume of negative pressure reservoir 148. In some embodiments, negative pressure reservoir 148 has a volume of at least 5,000 mm³ or at least 10,000 mm³. In some embodiments, negative pressure reservoir 148 is configured to maintain pressure at wound site 150 at least 50 mmHg below atmospheric pressure when tube 104 is detached from the negative pressure source. In some embodiments, negative pressure reservoir 148 maintains the pressure at wound site 150 at least 50 mmHg below atmospheric pressure for at least 24 hours when tube 104 is detached from the negative pressure source. In some embodiments, negative pressure reservoir 148 is configured to prevent pressure at wound site 150 from changing by more than 75 mmHg within a twelve hour period when tube 104 is detached from the negative pressure source.

### Experimental Test Results

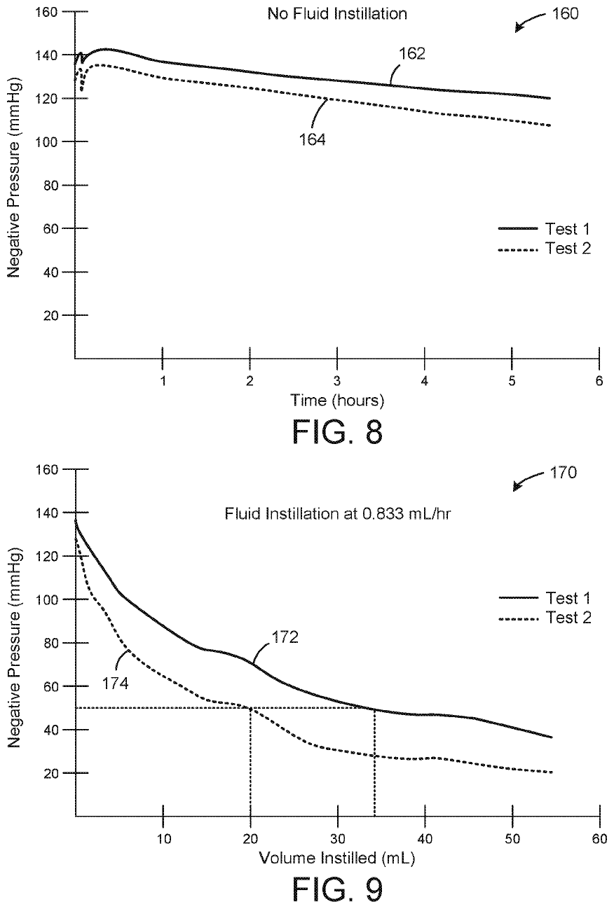

Referring now to FIGS. 8-9, a pair of graphs 160 and 170 illustrating experimental test results for NPWT system 100 and wound dressing 110 are shown, according to an exemplary embodiment. Both graphs 160 and 170 illustrate the ability of wound dressing 110 to maintain negative pressure under typical wound treatment conditions. In both experiments, wound dressing 110 was sealed to a surface and tube 104 was attached to pump unit 106. Pump unit 106 was operated to draw air out of wound dressing 110 and tube 104, thereby applying a negative pressure of approximately 130-140 mmHg relative to the pressure of the local environment (e.g., the atmosphere) outside wound dressing 110. Tube 104 was then disconnected from pump unit 106 and the pressure within wound dressing 110 was monitored to determine how well wound dressing 110 holds the negative pressure.

Graph 160 plots the results of a dry experiment in which no fluids were instilled to wound dressing 110. Line 162 illustrates the results of a first test, whereas line 164 illustrates the results of a second test. In the first test, the negative pressure within wound dressing 110 dropped from approximately 140 mmHg to approximately 120 mmHg over a time period of almost six hours. In the second test, the negative pressure within wound dressing 110 dropped from approximately 130 mmHg to approximately 110 mmHg over a time period of almost six hours. At this rate of decline, it would take significantly longer than twelve hours (e.g., 20-24 hours) for the negative pressure within wound dressing 110 to drop below 50 mmHg, which is considered therapeutic for NPWT. Accordingly, wound dressing 110 would only need to be changed or recharged (i.e., by reconnecting pump unit 106 and removing more air) once or twice per day to maintain the negative pressure at a therapeutic level.

Graph 170 plots the results of a wet experiment in which fluid was instilled to wound dressing 110 at a rate of 0.833 mL/hr. Line 172 illustrates the results of a first test, whereas line 174 illustrates the results of a second test. In the first test, the negative pressure within wound dressing 110 dropped from approximately 140 mmHg to approximately 50 mmHg after approximately 34 mL of fluid was instilled over a time period of approximately 40 hours. In the second test, the negative pressure within wound dressing 110 dropped from approximately 130 mmHg to approximately 50 mmHg after approximately 20 mL of fluid was instilled over a time period of approximately 24 hours. In both tests, the negative pressure within wound dressing 110 remained above 50 mmHg for at least 24 hours. Accordingly, wound dressing 110 would only need to be changed or recharged (i.e., by reconnecting pump unit 106 and removing more air) once per day to maintain the negative pressure at a therapeutic level.

### Configuration of Exemplary Embodiments

The construction and arrangement of the elements of the wound dressing and negative pressure wound therapy system as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements.

The elements and assemblies may be constructed from any of a wide variety of materials that provide sufficient strength or durability, in any of a wide variety of colors, textures, and combinations. Additionally, in the subject description, the word “exemplary” is used to mean serving as an example, instance, or illustration. Any embodiment or design described herein as “exemplary” is not necessarily to be construed as preferred or advantageous over other embodiments or designs. Rather, use of the word “exemplary” is intended to present concepts in a concrete manner. Accordingly, all such modifications are intended to be included within the scope of the present disclosure. Other substitutions, modifications, changes, and omissions may be made in the design, operating conditions, and arrangement of the preferred and other exemplary embodiments without departing from the scope of the appended claims.

The order or sequence of any process or method steps may be varied or re-sequenced according to alternative embodiments. Any means-plus-function clause is intended to cover the structures described herein as performing the recited function and not only structural equivalents but also equivalent structures. Other substitutions, modifications, changes and omissions may be made in the design, operating configuration, and arrangement of the preferred and other exemplary embodiments without departing from the scope of the appended claims.

1. A wound dressing comprising:
a cover layer sealable to skin surrounding a wound site and comprising a port extending through the cover layer;
a tube comprising a first end coupled to the cover layer via the port and a second end comprising an in-line connector configured to releasably attach the tube to a negative pressure source and detach the tube from the negative pressure source; and
a one-way valve located along the tube between the first end and the second end and configured to:
allow fluid flow through the tube in a first direction from the first end to the second end; and
prevent fluid flow through the tube in a second direction from the second end to the first end.

2. The wound dressing of claim 1, wherein the one-way valve is located within the in-line connector at the second end of the tube.

3. The wound dressing of claim 1, wherein the one-way valve is configured to maintain negative pressure within the tube when the tube is detached from the negative pressure source.

4. The wound dressing of claim 1, further comprising a filter configured to prevent liquid within the tube from reaching the one-way valve.

5. The wound dressing of claim 4, wherein the filter is located within the in-line connector at the second end of the tube.

6. The wound dressing of claim 1, further comprising a hydrophobic filter configured to retain liquid within the wound dressing, wherein the fluid flow through the tube is airflow.

7. The wound dressing of claim 1, further comprising a pressure indicator configured to measure a pressure within the tube or at the wound site.

8. (canceled)

9. (canceled)

10. The wound dressing of claim 1, wherein an internal volume of the tube between the one-way valve and the cover layer defines a negative pressure reservoir fluidly connected with the wound site and configured to stabilize changes in pressure at the wound site.

11. The wound dressing of claim 10, wherein the negative pressure reservoir within the tube has a volume of at least 5,000 mm³.

12. (canceled)

13. The wound dressing of claim 10, wherein the negative pressure reservoir is configured to maintain pressure at the wound site at least 50 mmHg below atmospheric pressure when the tube is detached from the negative pressure source.

14. (canceled)

15. The wound dressing of claim 10, wherein the negative pressure reservoir is configured to prevent pressure at the wound site from changing by more than 75 mmHg within a 12 hour period when the tube is detached from the negative pressure source.

16. (canceled)

17. (canceled)

18. (canceled)

19. (canceled)

20. The wound dressing of claim 1, wherein the tube is configured to configured to retract to form a substantially planar coil when not connected to the negative pressure source.

21. The wound dressing of claim 1, wherein the tube is pre-coiled such that the tube forms a substantially planar coil in the absence of external force applied to the tube.

22. The wound dressing of claim 1, wherein a cross-section of the tube is substantially rectangular.

23. The wound dressing of claim 1, wherein the tube has a wall thickness that provides sufficient rigidity to prevent collapse of the tube when a pressure within the tube is at least 100 mmHg below atmospheric pressure.

24. A negative pressure wound therapy system comprising:
a negative pressure source;
a wound dressing sealable to skin surrounding a wound site;
a tube comprising a first end coupled to the wound dressing and a second end comprising an in-line connector configured to releasably attach the tube to the negative pressure source and detach the tube from the negative pressure source; and
a one-way valve located along the tube and configured to prevent fluid flow through the tube from the second end to the first end.

25. (canceled)

26. (canceled)

27. (canceled)

28. (canceled)

29. (canceled)

30. (canceled)

31. (canceled)

32. (canceled)

33. The negative pressure wound therapy system of claim 24, wherein an internal volume of the tube between the one-way valve and the wound dressing defines a negative pressure reservoir fluidly connected with the wound site and configured to stabilize changes in pressure at the wound site.

34. The negative pressure wound therapy system of claim 33, wherein the negative pressure reservoir within the tube has a volume of at least 5,000 mm³.

35. (canceled)

36. (canceled)

37. The negative pressure wound therapy system of claim 33, wherein the negative pressure reservoir is configured to maintain pressure at the wound site at least 50 mmHg below atmospheric pressure for at least 24 hours when the tube is detached from the negative pressure source.

38. (canceled)

39. (canceled)

40. The negative pressure wound therapy system of claim 24, wherein the tube has an inner diameter between 2 mm and 3 mm.

41. The negative pressure wound therapy system of claim 24, wherein the tube has an inner diameter between 3 mm and 4 mm.

42. The negative pressure wound therapy system of claim 24, wherein the tube has an outer diameter of at least 4 mm and a wall thickness of at least 0.75 mm.

43. (canceled)

44. (canceled)

45. (canceled)

46. (canceled)

47. A wound dressing comprising:
a cover layer sealable to skin surrounding a wound site and comprising a port extending through the cover layer;
a tube comprising a first end coupled to the cover layer via the port and a second end comprising an in-line connector configured to releasably attach the tube to a negative pressure source and detach the tube from the negative pressure source;
a one-way valve located within the in-line connector and configured to prevent fluid flow into the tube via the second end;
a filter located within the in-line connector and configured to prevent liquid within the tube from reaching the one-way valve; and
a pressure indicator located within the in-line connector and configured to measure a pressure at the second end of the tube.
